(19) United States
(12) Patent Application Publication
(10) Pub. No.: US 2010/0286553 A1
(43) Pub. Date: Nov. 11, 2010

(54) Devices and Methods for Screening of Vagal Nerve Stimulation

(76) Inventors: Claudio A. Feler, Memphis, TN (US); Scott F. Drees, McKinney, TX (US)

Correspondence Address: HAYNES AND BOONE, LLP;IP Section, 2323 Victory Avenue, Suite 700, Dallas, TX, 75219, US

(21) Appl. No.: 12/463,139

(22) Filed: May 8, 2009

(51) Int. Cl. A61B 5/05

(52) U.S. Cl. 600/554

(57) ABSTRACT

The present disclosure provides systems and methods for screening vagus nerve stimulation to determine the potential efficacy of permanent stimulation systems. In one aspect, the system includes temporary electrode assemblies adapted for temporary placement on or in the body adjacent the vagus nerve. In another aspect, a method is provided to place a stimulating electrode adjacent the posterior of the carotid sheath.

### BACKGROUND OF THE INVENTION

Epilepsy is characterized by a tendency to recurrent seizures that can lead to loss of awareness, loss of consciousness, and/or disturbances of movement, autonomic function, sensation (including vision, hearing and taste), mood, and/or mental function. The mean prevalence of active epilepsy (i.e., continuing seizures or the need for treatment) in developed and undeveloped countries combined is estimated to be 7 per 1,000 of the general population, or approximately 40 million people worldwide. Studies in developed countries suggest an annual incidence of epilepsy of approximately 50 per 100,000 of the general population. However, in other literature it is suggested that in developing countries this figure is nearly double at 100 per 100,000 of general population.

Epilepsy is often but not always the result of underlying brain disease. Any type of brain disease can cause epilepsy, but not all patients with the same brain pathology will develop epilepsy. The cause of epilepsy cannot be determined in a number of patients; however, the most commonly accepted theory posits that it is the result of an imbalance of certain chemicals in the brain, e.g., neurotransmitters. Children and adolescents are more likely to have epilepsy of unknown or genetic origin. The older the patient, the more likely it is that the cause is an underlying brain disease such as a brain tumor or cerebrovascular disease.

Trauma and brain infection can cause epilepsy at any age, and in particular, account for the higher incidence rate in developing countries. For example, in Latin America, neurocysticercosis (cysts on the brain caused by tapeworm infection) is a common cause of epilepsy; in Africa, AIDS and its related infections, malaria and meningitis, are common causes; in India, AIDS, neurocysticercosis and tuberculosis, are common causes. Febrile illness of any kind, whether or not it involves the brain, can trigger seizures in vulnerable young children, which seizures are called febrile convulsions. About 5% of such children go on to develop epilepsy later in life. Furthermore, for any brain disease, only a proportion of sufferers will experience seizures as a symptom of that disease. It is, therefore, suspected that those who do experience such symptomatic seizures are more vulnerable for similar biochemical/neurotransmitter reasons.

Studies in both developed and developing countries have shown that up to significant percentage of newly diagnosed children and adults with epilepsy can be successfully treated (i.e., complete control of seizures for several years) with anti-epileptic drugs. After two to five years of successful treatment, drugs can be withdrawn in a large portion of the children and of adult patients without the patient experiencing relapses. However, up to 30% of patients are refractory to medication. There is evidence that the longer the history of epilepsy, the harder it is to control. The presence of an underlying brain disease typically results in a worse prognosis in terms of seizure control. Additionally, partial seizures, especially if associated with brain disease, are more difficult to control than generalized seizures.

Vagus nerve stimulation is currently used as a therapy for refractory epilepsy, and studies have suggested that such stimulation may also be an efficacious therapy for tremor, depression, obesity, and gastroesophageal reflux disease (GERD). Currently available vagus nerve stimulators require a significant surgical procedure for placement and create the possibility of generating scar tissue adjacent the vagus nerve. Additionally, the pulse generator is battery-powered, which battery needs to be changed periodically, and the pulse generator may be uncomfortable and cosmetically unpleasing as well.

Patients suffering from tremor and other symptoms may undergo surgery to lesion a part of the brain, which may afford some relief. However, a lesion is irreversible, and it may lead to side effects such as dysarthria or cognitive disturbances. Additionally, lesions generally yield effects on only one side (the contralateral side), and bilateral lesions are significantly more likely to produce side effects. Other surgical procedures, such as fetal tissue transplants, are costly and unproven.

Patients suffering from epilepsy may undergo surgery to remove a part of the brain in which the seizures are believed to arise, i.e., the seizure focus. However, in many patients a seizure focus cannot be identified, and in others the focus is in an area that cannot be removed without significant detrimental impact on the patient. For example, in temporal lobe epilepsy, patients may have a seizure focus in the hippocampi bilaterally. However, both hippocampi cannot be removed without devastating impacts on long-term memory. Other patients may have a seizure focus that lies adjacent to a critical area such as the speech center.

As mentioned above, vagus nerve stimulation (VNS) has been applied with some success in patients with refractory epilepsy. In the existing procedure, an implantable pulse generator (IPG) is implanted in the patient's thorax, and an electrode lead is routed from the IPG to the left vagus nerve in the neck. Helix-shaped stimulation and indifferent electrodes are attached directly to the vagus nerve via an invasive surgical process that requires the carotid sheath to be fully exposed and excised to gain access to the vagus nerve. Based on some reported studies, approximately 5-15% of patients undergoing VNS are seizure-free, and an additional 30-40% of patients have a greater than 50% reduction in seizure frequency. The remaining patients receive little or no benefit from the implantation of the stimulation system.

Drawbacks of available VNS, such as size (of internal and/or external components), discomfort, inconvenience, and/or complex, risky, and expensive surgical procedures, has generally confined their use to patients with severe symptoms and the capacity to finance a surgery with unknown outcomes. Some side effects of VNS include voice alterations, cough, pharyngitis, and dyspnea.

Thus, there remains a need for a reliable screening method to determine stimulation efficacy as well as methods of lead placement. The present disclosure overcomes one or more shortcomings in the art.

### BRIEF SUMMARY OF THE INVENTION

The present invention relates to a method of screening for efficacy of vagus nerve stimulation to determine whether vagus nerve stimulation has an impact on patient symptoms. In one aspect, the method includes placing at least one temporary electrode adjacent the vagus nerve and securing the temporary electrode in position adjacent the vagus nerve. The electrode is energized to stimulate at least a portion of the vagus nerve and the patient is monitored to determine the response to the stimulation of the vagus nerve and whether the stimulation had a beneficial effect on the patient. After the screening period is complete, the temporary electrode is removed from the patient. In one aspect, the temporary electrode is positioned on the patient's skin adjacent the vagus nerve and secured using adhesive. In an alternative aspect, at least one temporary electrode is positioned beneath the patient's skin.

In another aspect, the method of screening for efficacy of vagus nerve stimulation includes forming an opening in the patient's skin and positioning a temporary electrode beneath the skin of the patient. In one aspect, the method includes performing a blunt needle stick adjacent the carotid sheath of the patient. In one form, a portion of the electrode is imaged with imaging equipment to monitor its position in the patient. In a further aspect, contrast media is applied in the vascular system while positioning the electrode to more fully visualize the anatomic structures in relation to the electrode position. During the method, the temporary electrode is advanced along the carotid sheath substantially parallel to the vagus nerve and temporarily secure in position within the patient. In one form, the method includes advancing the electrode to a position along the exterior of the carotid sheath. In a further aspect, the electrode is positioned posterior to the vagus nerve and exterior to the carotid sheath. In another form, the method includes advancing the temporary electrode within the carotid sheath. In still a further aspect, lead wires extending from the temporary stimulation electrodes extend outside of the patient's skin and are connected to a stimulation control unit. In an alternative form, the temporary electrode is part of a self contained stimulation generator system and no lead wires extend outside of the patient's skin. After the screening period, the temporary electrode may be removed from the patient.

In still another aspect, the method of screening for efficacy of vagus nerve stimulation includes creating an incision above the clavicle and forming a pocket generally parallel to and outside of the carotid sheath of the patient. A temporary lead with electrodes is then inserted into the pocket with the electrodes positioned to engage at least a portion of the carotid sheath facing the vagus nerve. In one form, the method includes inserting the temporary lead into the patient in an insertion configuration and positioning the temporary lead electrodes to extend about at least a portion of the carotid sheath by deploying the temporary lead to a stimulating configuration adjacent the carotid sheath. In one aspect, the step of deploying includes orienting the electrodes toward the vagus nerve and positioning an antimigration and/or antirotation stabilization member associated with the lead to maintain the electrodes position adjacent the carotid sheath and directed toward the vagus nerve. In still a further aspect, lead wires extending from the temporary stimulation electrode extend outside of the patient's skin and are connected to a stimulation control unit. In an alternative form, the temporary electrode is part of a self contained stimulation generator system and no lead wires extend outside of the patient's skin.

In yet a further aspect, the present invention includes a kit for performing temporary trial stimulation of the vagus nerve to screen. In one form, the kit includes a base member having thereon one or more electrodes, the base member is configured for mechanical coupling to a releasable anchoring system that can be attached to the patient and be atraumatically removed from the patient. In one aspect, the releasable anchoring system can be manually removed from the patient without surgical access to the electrode. In still another aspect, a movable stabilization member is provided adjacent the one or more electrodes and acts to orient the electrodes toward the vagus nerve. In another aspect, the kit includes a retrieval instrument to move the stabilization member to a collapsed condition so the electrode may be removed from the patient.

In still a further aspect, the disclosure includes a method of screening for non-responding patients that are not responsive to vagus nerve stimulation therapies. In a further aspect, the method identifies the magnitude of the beneficial effect of patients' responsive vagus nerve stimulation.

Further aspects, forms, embodiments, objects, features, benefits, and advantages of the present invention shall become apparent from the detailed drawings and descriptions provided herein.

### BRIEF DESCRIPTION OF THE SEVERAL VIEWS OF THE DRAWINGS

The above and other aspects of the present invention will be more apparent from the following more particular description thereof, presented in conjunction with the following drawings wherein:

FIG. 1 illustrates a prior art implantation of a vagus nerve stimulation system.

FIGS. 2A and 2B illustrate application of a temporary external vagus nerve stimulation system according to one aspect of the present invention.

FIGS. 3A-3C illustrate techniques for temporary placement of internal electrodes according to another aspect of the present invention.

FIGS. 4A and 4B illustrate a technique for lead placement posterior to the carotid sheath according to another aspect of the present invention.

FIG. 5 illustrates an externally applied temporary stimulation electrode panel according to another aspect of the present invention.

FIGS. 6A and 6B illustrate a cylindrical stimulation electrode assembly.

FIGS. 7A and 7B illustrate a cylindrical stimulation electrode assembly with shielded stimulation electrodes and an anti-rotation assembly.

FIGS. 8A-8D illustrate a further embodiment of a shielded stimulation electrode assembly with an anti-rotation assembly.

FIGS. 9A and 9B illustrate a paddle electrode lead according to another aspect of the present invention.

FIG. 10 illustrates a further stimulation lead configured for use in accordance with the present invention.

FIGS. 11A and 11B illustrate stimulation capsules configured for use in accordance with the present invention.

### DETAILED DESCRIPTION OF THE INVENTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

In accordance with the teachings of the present disclosure and as discussed in more detail presently, screening for efficacy of electrical stimulation at one or more locations along the vagus nerve 100 and/or its branches is provided to evaluate the efficacy of permanent stimulation implantation to treat, control, and/or prevent epilepsy, metabolic disorders (including obesity), mood disorders (including depression and bipolar disorder), anxiety disorders (including generalized anxiety disorder and obsessive-compulsive disorder), chronic pain (including visceral pain, neuropathic pain and nociceptive pain), gastrointestinal disorders (including gastroesophageal reflux disease (GERD), fecal dysfunction, gastrointestinal ulcer, gastroparesis, and other gastrointestinal motility disorders), hypertension, cardiac disorders (including tachycardia, bradycardia, other arrhythmias, congestive heart failure, and angina pectoris), psychotic disorders (including schizophrenia), cognitive disorders, dementia (including Alzheimer's disease, Pick's disease, and multi-infarct dementia), eating disorders (including anorexia nervosa and bulimia), sleep disorders (including insomnia, hypersomnia, narcolepsy, and sleep apnea), endocrine disorders (including diabetes), movement disorders (including Parkinson's disease and essential tremor), and/or headache (including migraine and chronic daily headache). A temporary electrode may be positioned transdermally or percutaneously adjacent the vagus nerve. Although the drawings illustrate stimulation methods associated with the left vagus nerve, it is intended that similar trial and screening techniques can be applied bilaterally or separately to the right vagus nerve or any branches thereof to screen the patient for the intended beneficial result expected from permanent implantation of a stimulation electrode.

Trial stimulation of the vagus nerve may occur distal to (i.e., below) the superior cervical cardiac branch, or distal to both the superior cervical cardiac branch and the inferior cervical cardiac branch, and may, for instance, be applied to the left vagus nerve. Stimulation of the left vagus nerve distal to the superior cervical cardiac branch and/or the inferior cervical cardiac branch does not pose the cardiac risks that can be associated with vagus nerve stimulation applied proximal to one or both of these nerve branches. Alternatively, some patients may benefit from vagus nerve stimulation applied distal to the thoracic cardiac branch.

As used herein, trial stimulation screening of the vagus nerve may include stimulation of the vagus nerve and/or one or more of its branches. For instance, to relieve sleep disorders (such as insomnia, hypersomnia, narcolepsy, sleep apnea, and the like), the vagus nerve may be stimulated. More specifically, one or more of the pharyngeal branch of the vagus nerve, the superior laryngeal branch of the vagus nerve, the pharyngeal plexus (not shown), the left and/or right recurrent laryngeal branch of the vagus nerve, and/or other branches of the vagus nerve may be stimulated to relieve sleep disorders. As another example, the vagus nerve may be stimulated to relieve gastrointestinal disorders (such as including gastroesophageal reflux disease (GERD), fecal dysfunction, gastrointestinal ulcer, gastroparesis, and other gastrointestinal motility disorders). More specifically, one or more of the gastrointestinal branches of the vagus nerve, such as the anterior gastric branch of the anterior vagal trunk, the right gastric plexus, and/or the left gastric plexus may be stimulated to relieve gastrointestinal disorders. As yet another example, to relieve endocrine disorders (including diabetes), the vagus nerve may be stimulated. More specifically, one or more branches innervating the pancreas, such as the anterior superior and anterior inferior pancreaticoduodenal plexus, the posterior pancreaticoduodenal plexus (not shown), the inferior pancreaticoduodenal plexus, or the like may be stimulated to relieve endocrine disorders.

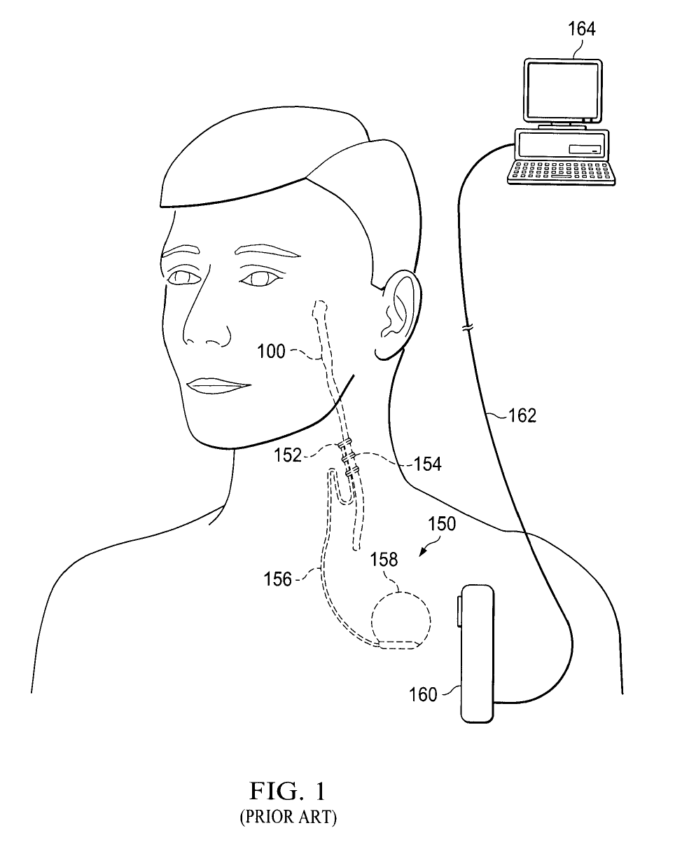

Referring now to FIG. 1, there is shown a prior art surgical placement of a permanent electrode assembly for stimulation of the vagus nerve. The stimulation system 150 includes electrodes 152 and 154 positioned within the carotid sheath and wrapped around the vagus nerve to anchor their position along the vagus nerve 100. This system requires direct attachment to the vagus nerve to obtain efficient stimulation and to anchor the position of the electrodes along the vagus nerve. The procedure of placing the electrodes on the vagus nerve is a delicate operation and requires significant exposure and access through the carotid sheath. Lead wire 156 is snaked under the skin within the patient and attached to an implantable pulse generator (“IPG”) 158. The IPG may be coupled through the patient's skin by transducer 160. Transducer 160 is connected to a computer 164 by wire 162. In this manner, the IPG may be programmed to adjust the stimulation signals as needed.

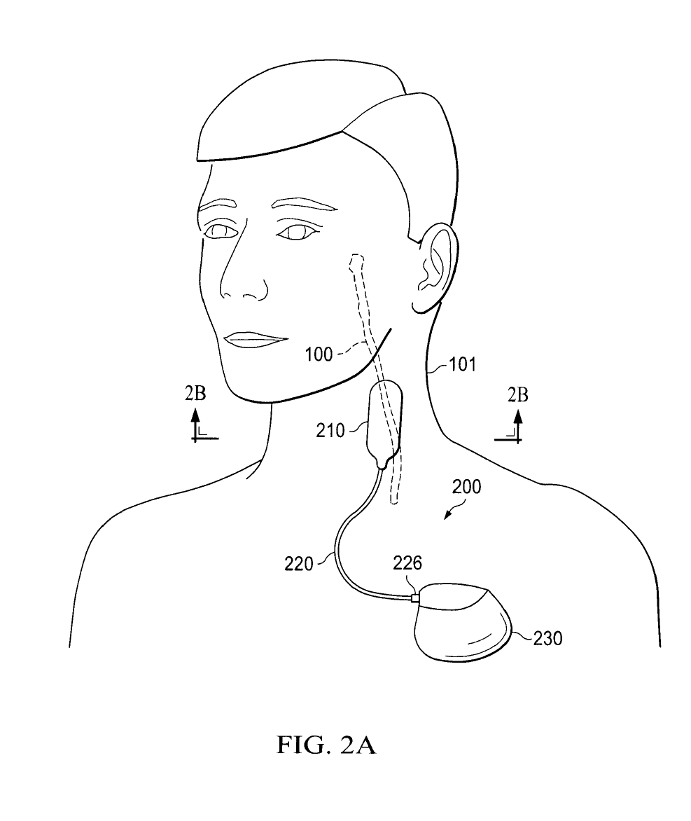

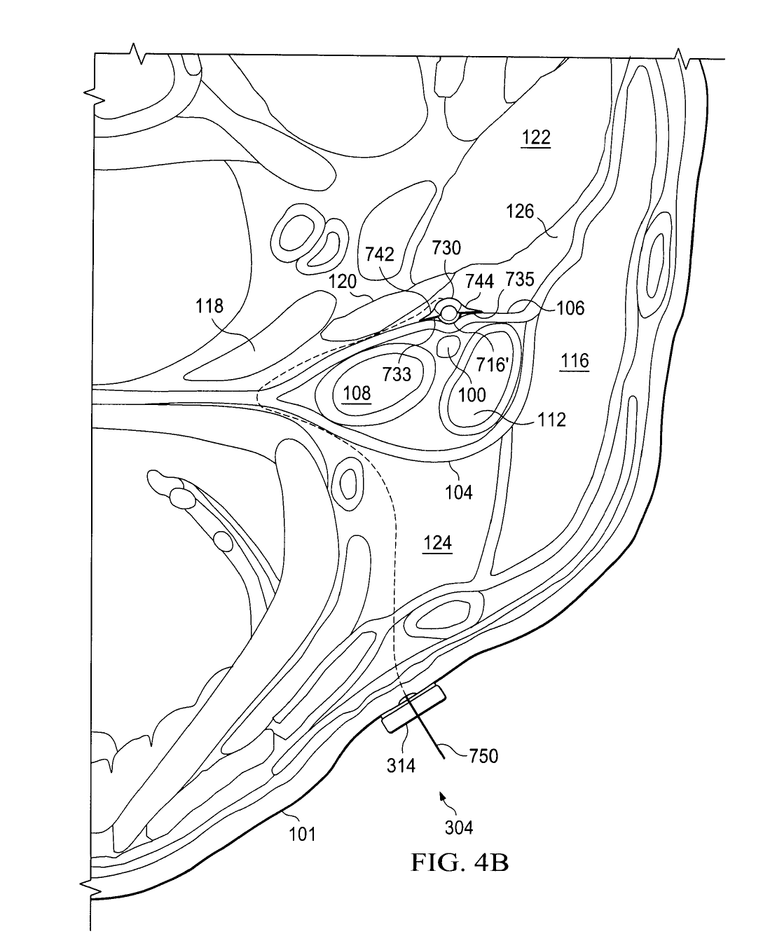

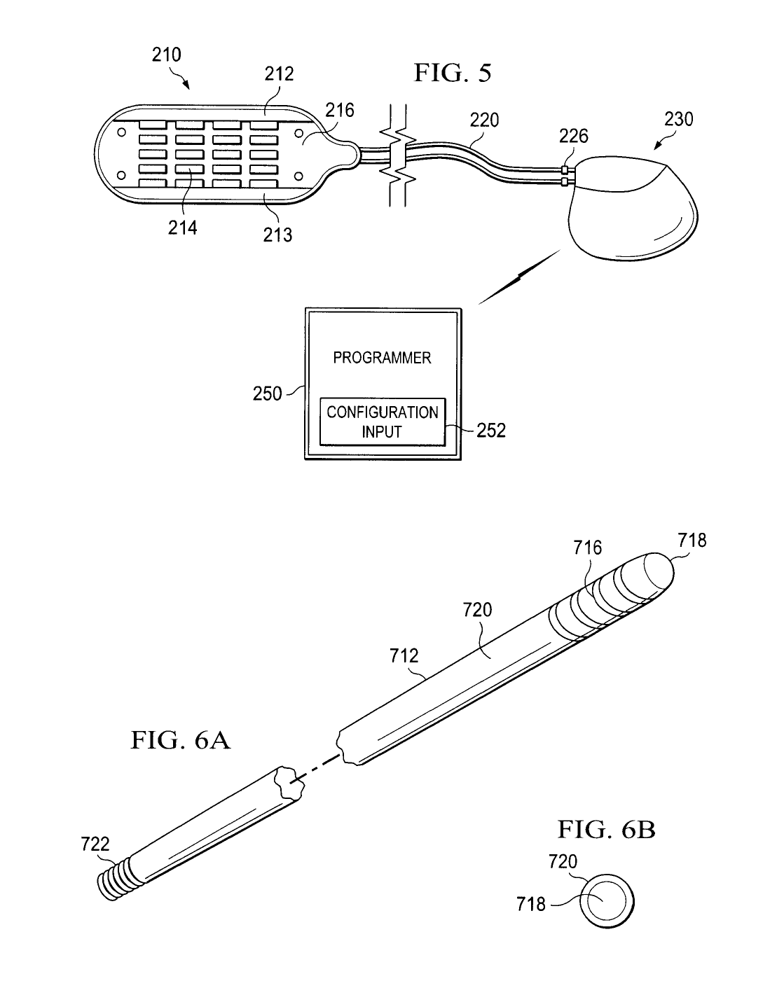

Referring now to FIGS. 2A, 2B, and 5 there is shown a temporary electrode stimulation assembly according to one aspect of the present disclosure. The temporary electrode stimulation assembly 200 includes an electrode array 210 connected via a series of leads 220 through coupling 226 to an external pulse generator 230. The electrode array includes a body portion 216 connecting a series of electrodes 214. The electrodes are electrically coupled to the leads 220. The electrode array 210 further includes adhesive layers 212 and 213 formed on its underside surface facing the patient. As shown in FIG. 5, the pulse generator can be controlled wirelessly by programmer 250 having a configuration input 252.

The electrode assembly 200 is utilized for vagus nerve trialing procedures in the following manner. Adhesive layer 212 is exposed and the healthcare provider positions the electrode array 210 to extend along the skin substantially in alignment with the carotid sheath. Pressure is applied to the electrode array 210 to cause adhesive layer 212 to releasable adhere to the patient's skin. The lead wires 220 are then coupled to the pulse generator 230 by connection through coupling 226. Once the electrode array has been positioned and anchored, the pulse generator is controlled to provide one or more pulse to the electrode array 210. The patient is monitored to determine if sufficient energy is reaching the vagus nerve to cause the desired stimulation. If not, the energy applied may be increased by controlling the pulse generator to create a higher energy output. In the alternative or in combination, the electrode array 210 may be removed from the patient and repositioned on the skin to better align the electrodes with the vagus nerve 100 positioned within the carotid sheath. Pressure may be applied to the electrode array 210 to cause the adhesive layer 212 to adhere to the skin to secure the array in position. Once sufficient energy is reaching the vagus nerve 100 to cause the desired stimulation, the screening method may proceed. If the patient has been monitored for seizure frequency and intensity to establish a baseline before attachment of the temporary electrodes, then the pulse generator may be started to begin stimulation. If no baseline for the patient is available, then the patient will be observed for an initial period to establish a seizure baseline. Once the baseline is established, the pulse generator will be controlled to deliver the desired stimulation.

The present disclosure contemplates use of several stimulation strategies depending on patient symptoms and professional judgment. In one aspect, the pulse generator is controlled to deliver constant stimulation to the electrode array 210 and thereby to the vagus nerve. In another strategy, the pulse generator is controlled to deliver intermittent pulses to the electrode array 220 to periodically stimulate the vagus nerve on a set schedule. In yet another strategy, the pulse generator delivers reactive pulse based on the sensed onset of a seizure. Still further, the pulse generator can be controlled manually by the patient or by an observer. Additionally, the method may include the attachment of one or more sensors to detect evidence, such as electrical signals, of the onset or occurrence of seizure activity. The sensed data is analyzed to determine the seizure onset or occurrence and the pulse generator is controlled in response to the sensed data to generate pulses to stimulate the vagus nerve. Regardless of the stimulation strategies utilized, the patient's response to vagus nerve stimulation is observed. The patient's seizure activity during one or more of the vagus nerve stimulation strategies is compared to the previously acquired baseline seizure activity. From this comparison, it can be determined whether the patient is likely to benefit from an implanted pulse generator electrically connected to leads fixed directly to the vagus nerve as shown in FIG. 1. Further, the data received during the screening period and its comparison to the baseline information can be used to determine the expected magnitude of patient relief that can be expected from a permanently placed lead and stimulation system.

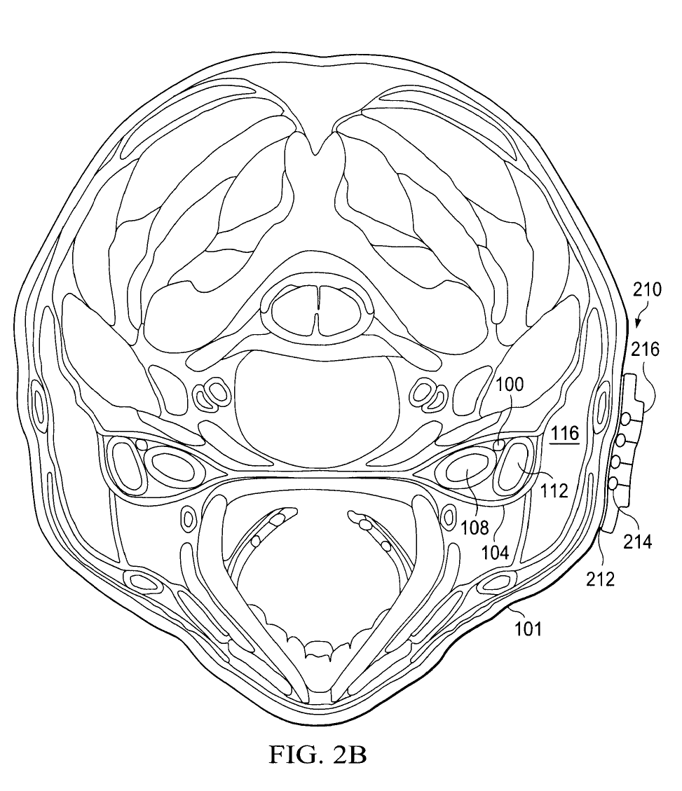

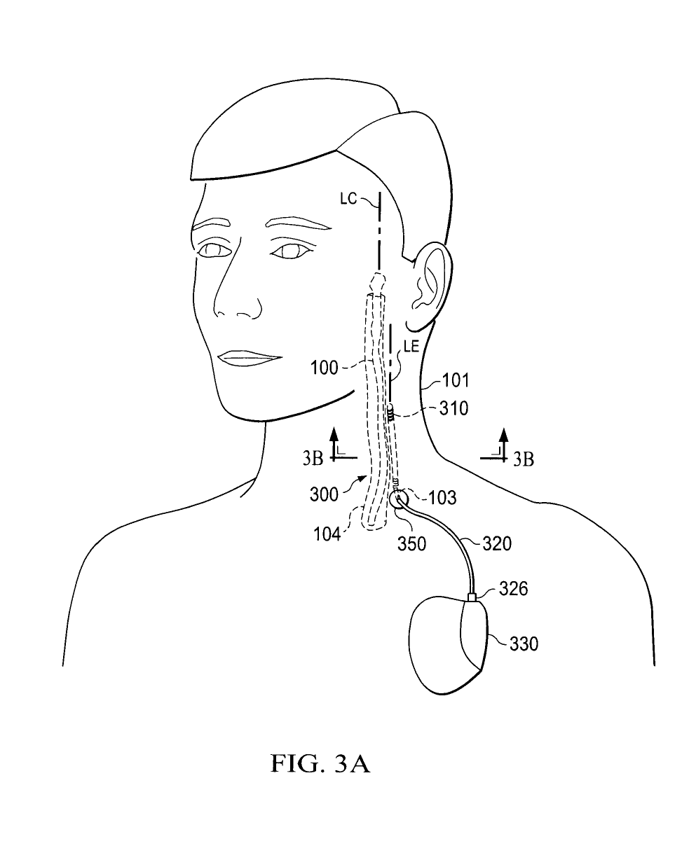

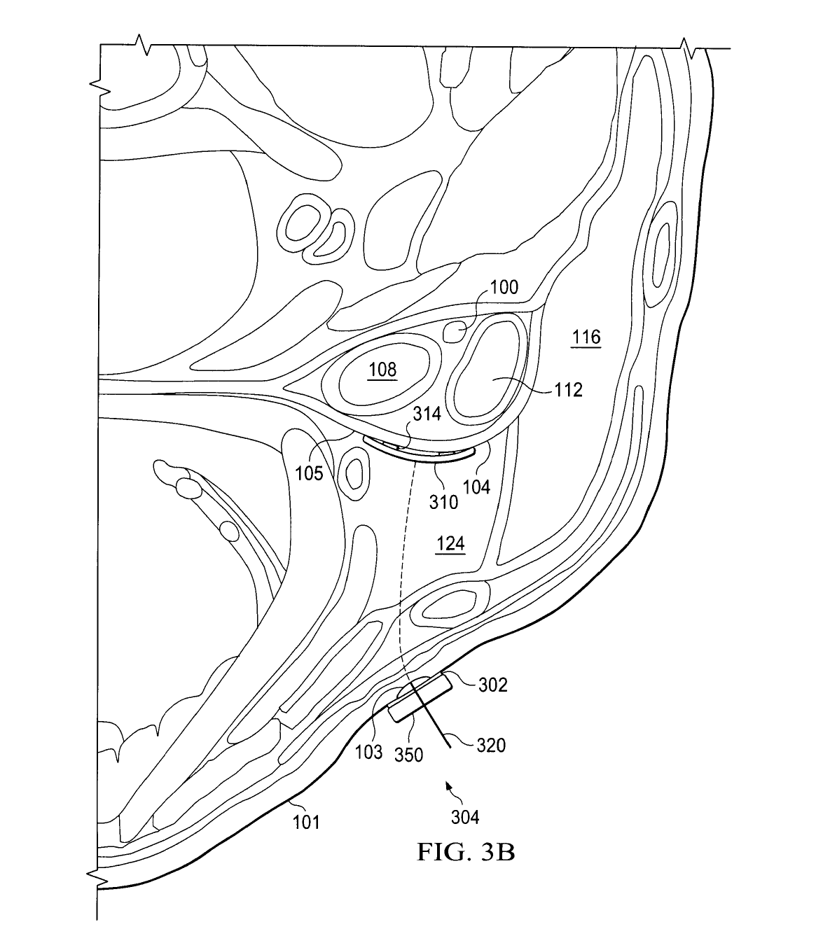

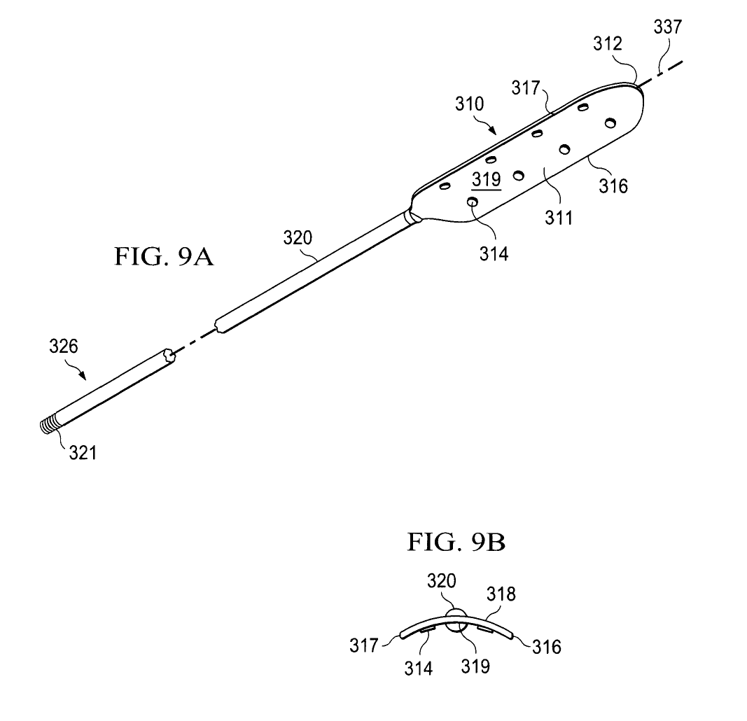

Referring now to FIGS. 3A and 3B, there is shown an alternative embodiment according to another aspect of the present disclosure. In this screening method, a temporary electrode 310 is placed beneath the skin 101 of the patient through an opening 103 formed in the skin. With reference to FIGS. 9A and 9B, electrode 310 is a percutaneously implantable lead having eight electrodes 314 extending from a distal tip 312. The electrode lead portion 311 is generally. That is upper and lower surfaces 318 and 319 are substantially larger than the surface area of the side wall surfaces 316 and 317. More specifically, upper and lower surfaces 318 and 319 have a greater width transverse to the longitudinal axis 337 than side walls 316 and 317. Electrode 310 is a type of paddle electrode. Still further, the electrode surface 319 is substantially a concave surface while the opposing surface 318 is a substantially convex surface. The electrode lead portion 311 is connected to an elongated lead 320 that includes electrical conductors connected to each electrode 314. As discussed in relation to the method of placement, it is contemplated that the concave surface 319 is configured to face the vagus nerve with exposed electrodes 314 as shown, while surface 318 is formed of a non-conductive material, or includes an insulating coating, to inhibit unwanted stimulation of body structures adjacent surface 318. Referring back to FIG. 3A, individual lead wires 321 of lead 320 are connectable to pulse generator 330 by coupling 326.

A surgical procedure for implantation of the temporary electrode 310 will be explained with reference to FIGS. 3A and 3B. Utilizing image guidance, a healthcare provider performs a blunt needle stick to form opening 103 in the skin below a target placement site next to the carotid sheath 104. One or more sequential dilators are passed through opening 103 to form an enlarged passage along the carotid sheath 104. A percutaneous lead such as temporary electrode 310 is delivered through opening 103 into the passage along the carotid sheath substantially parallel to the vagus nerve. In one aspect, insertion of the temporary electrode 310 is accomplished without the use of a lead blank or a blunt sheath. In an alternative aspect, the temporary electrode 310 is supported during insertion using a lead blank or a blunt sheath. In this technique, the electrode is positioned using the blunt sheath and the blunt sheath is withdrawn leaving the temporary electrode 310 in position adjacent the carotid sheath 104 and the vagus nerve 100. Once the temporary electrode 310 is positioned adjacent the vagus nerve, a test may be performed to confirm that when energized the electrode properly stimulates the vagus nerve. If necessary, the temporary electrode 310 may be repositioned to obtain the necessary stimulation of the vagus nerve. Once properly positioned, the position of the temporary electrode 310 is maintained in the position, at least in part, by securing lead 320 to the skin 101 by an anchor 350. The exterior surface of the electrode 310 also assists in securing its position within the patient. The electrode surface 319 engages the carotid sheath 104 on the anterior surface 105. Similarly, the electrode surface 318 engages the adjacent tissue, such as fatty tissue 124 shown in the illustration. In one aspect, the skin anchor 350 is a bacteriostatic ring including an anti-bacterial compound to inhibit infection through skin opening 103. Such a bacteriostatic ring includes an adhesive layer 302 to join to the skin as well as a central opening to surround lead wire 320. Once the lead is positioned, the stimulation protocols described above may be followed to evaluate patient response to vagus nerve stimulation.

In still a further aspect, temporary electrode 3 10 may include a lubricious coating. The lubricious coating may help ease insertion of the electrode into the position shown in FIG. 3B. Further, after a period of trialing the stimulation protocols for the vagus nerve, the lubricious coating may ease retrieval of the temporary electrode 310. In one method, the temporary electrode 310 is removed from the body by pulling on lead 320 outside of the skin 101 until the electrode 310 exists opening 103.

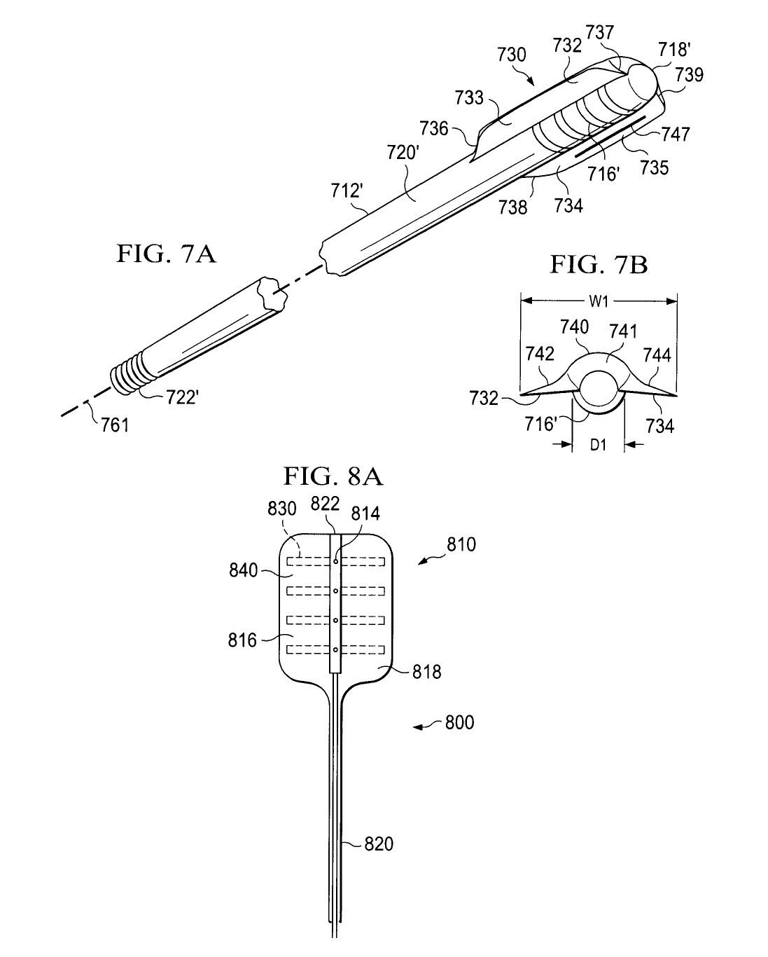

In an alternative surgical approach, the procedure includes gaining percutaneous surgical access through opening 307 to the interior of the carotid sheath 104 as shown in FIG. 3C. Once inside the carotid sheath, the electrode is moved cephalad, toward the head, while within the carotid sheath 104 and is passed between the jugular vein 112 and the carotid artery 108. As shown in FIG. 3C, a temporary electrode 712 is positioned within the carotid sheath 104 immediately adjacent the vagus nerve 100. It is contemplated that the temporary electrode 712 would include a substantially smooth outer surface to atraumatically engage an exterior surface of the vagus nerve 100. In this manner, the temporary electrode 712 can be positioned to abuttingly engage the exterior of the vagus nerve and can be removed without disturbing or damaging the vagus nerve 100 or adjacent vessels. Exemplary electrodes 712 and 712′ are shown in FIGS. 6A-7B. These electrodes will be further described below.

In still a further surgical approach for temporary lead placement, a small transverse incision a few centimeters above the clavicle is made in the patient's skin 101. Utilizing a blunt instrument or a finger, a pocket is formed between the interior of the patient's skin and the carotid sheath 104 to make room for a paddle electrode such as electrode 310. As shown in FIG. 3B, paddle electrode 310 is positioned in the pocket with concave side 319 positioned adjacent to the carotid sheath 104 and with the longitudinal axis 337 of the body positioned in substantial parallel alignment with the longitudinal axis of the adjacent vagus nerve 100. The lead 320 is secured to the skin as described above and stimulation would proceed as previously described.

Referring now to FIGS. 6A and 6B, there is shown a stimulation lead adapted for use in the screening technique disclosed herein. Stimulation lead 712 includes a shaft 720 and a coupling connection end 722. Adjacent the leading end 718 of the cylindrical lead body 720 are a series of electrodes 716. In the illustrated embodiment, there are four cylindrical electrodes spaced apart along the shaft adjacent the distal end 718. In this embodiment, the electrodes are cylindrical such that they can stimulate in a 360 degree pattern around the longitudinal axis of the lead body 720.

Referring now to FIGS. 7A and 7B, there is shown a stimulation lead adapted for use in the screening technique disclosed herein. Stimulation lead 712′ includes a shaft 720′ and a coupling connection end 722′ substantially similar to the design of FIG. 6. Electrode assembly 730 includes an additional stabilization member to orient the rotational position of electrodes 716′ about longitudinal axis 761 toward the vagus nerve and maintain the position of the electrodes along the vagus nerve once properly positioned. Specifically, the electrode assembly 730 includes a stabilization member having a first wing 732 and a second wing 734 extending laterally way from the longitudinal axis 761 of lead body 720′ adjacent the electrodes 716′. Wing 732 includes a trailing taper 736 extending distally from the lead body 720′ and a leading taper 737 extending proximally from lead tip 718′ and defining therebetween a front tissue engagement surface 733. Similarly, wing 734 includes a trailing taper 738 extending distally from the lead body 720′ and a leading taper 739 extending proximally from lead tip 718′ and defining therebetween a front tissue engagement surface 735. As shown in FIG. 7A, the length of the first and second wings along the longitudinal axis 761 of the lead body 720′ is slightly longer than the length of the electrode array on the lead body. In the illustrated embodiment, the length of the wings is selected to be greater than the electrode array to provide an electrical shield to surrounding tissues. In an alternative embodiment, this length can be varied to be less than the electrode array length. Referring to FIG. 7A, there is shown a radiopaque marker 747 disposed in wing 734. The material forming the stabilization wings is radiolucent. It will be understood that the marker will assist the surgeon in placing the electrode assembly in the correct orientation in the body.

Referring now to FIG. 7B, there is shown an end view of the electrode assembly 730. As illustrated, the lead body adjacent electrode 716′ had a diameter D1. The wings 732 and 734 of the anti-rotation stabilization member extend outward transverse to the longitudinal axis 761 of the lead body to define a stabilization member width W1. In one aspect W1 is at least twice is great at D1. In the illustrated aspect W1 is three times larger than D1. Electrode assembly 730 includes an insulating barrier 741 with a rear facing surface 740. The insulating barrier 741 defines a forward stimulation area on the electrode assembly and an opposing rearward insulation area. In the illustrated embodiment, the forward stimulation area extends approximately 180 degrees around the longitudinal axis 761 of the lead body 720′. The amount of circumferential lead exposure can be adjusted to have less or more exposure to match specific patient anatomy or vessel structure. The electrode assembly 730 includes rear surfaces 742 and 744 on wings 732 and 734, respectively. In one aspect, the wings 732 and 734 are integrally formed with insulating barrier 741 of a non-conductive material. It is contemplated that, without limitation to other materials, the barrier can include a polymer such as polyethylene, PTFE, polyurethane or other polymers used to form leads and catheters. In yet a further embodiment, insulting barrier includes an internal bladder that extends, at least partially, within wings 732 and 734. The internal bladder is connected to a filling cannula (not shown) that extends along the length of lead body 720′. The electrode assembly 730 can be inserted with the bladder in a collapsed state and then fluid injected through the filling cannula to expand the bladder. Expansion of the bladder will urge wings 732 and 734 to deploy and prevent collapse of the wings to help maintain the position of the electrode assembly adjacent the vagus nerve. Still further, the exterior surfaces 733, 735, 740, 742, and/or 744 may be coated with a lubricious coating to ease insertion through the insertion cannula as well as placement and expansion of the wings in the body. In still a further aspect, the lubricious coating maintains lubricity for several weeks such that the lead 712′ can be easily removed from the surrounding tissue with minimal tissue damage or irritation.

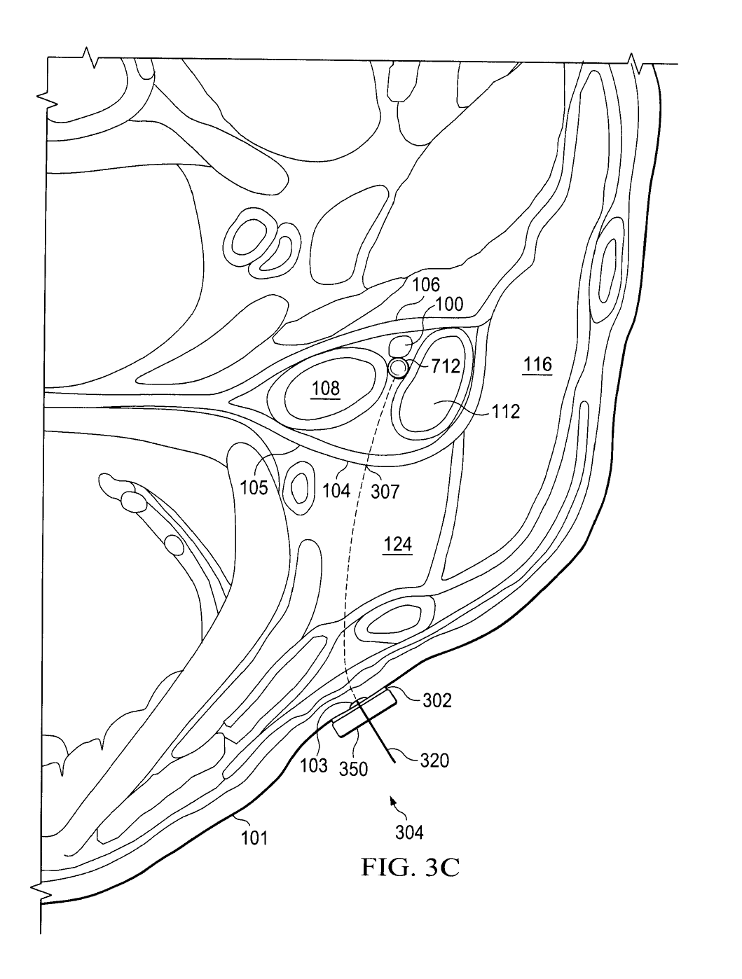

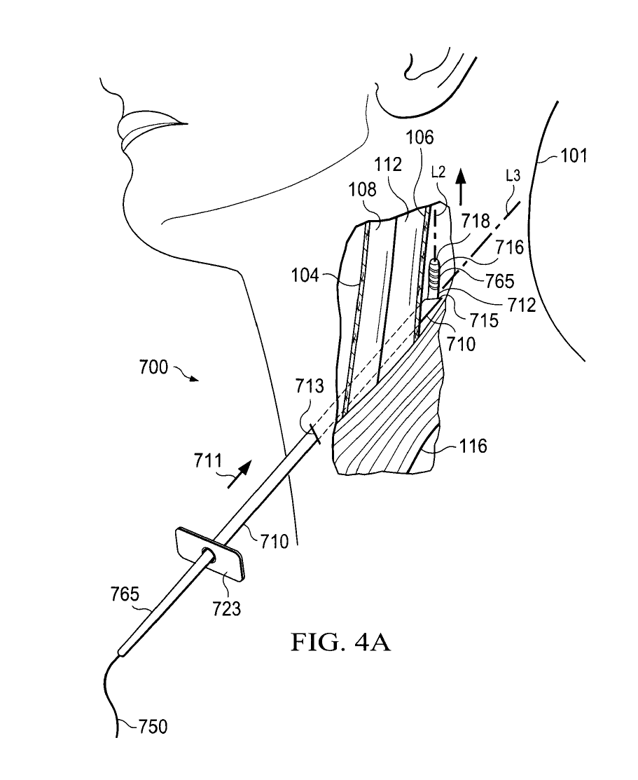

Referring now to FIGS. 4A and 4B, there is illustrated aspects of an additional surgical technique associated with the disclosed screening method. In the illustrated technique, a stimulating electrode assembly 716′ is positioned along the posterior surface 106 of the carotid sheath 104 adjacent the vagus nerve 100. In one aspect, the patient is positioned as shown in FIG. 4A. A healthcare provider identifies the carotid artery 108 adjacent the carotid tubercle of the C6 spinal level. With manual finger pressure on the skin 101, the carotid sheath 104 is swept laterally to create an access corridor toward the anterior facet of the cervical vertebra. With a slight upward cant, a needle 710 is used to pierce the skin 101 at opening 713. Using fluoroscopic guidance, the needle is pushed posteriorly in the direction of arrow 711 until the tip is positioned adjacent to an anterior facet of the cervical vertebra. Fluoroscopic guidance can include anterior-posterior fluoroscopy and/or lateral fluoroscopy to assist the healthcare provider in determining the relative position of the tip of the needle in relation to the patient's anatomical structures. Additionally, intravenous contrast media may be injected to highlight the blood vessels during the procedure. In one aspect, the tip of the needle is advanced until it engages the vertebral bone. This can be confirmed by fluoroscopic visualization and/or tactile feedback through the needle to the surgeon. In an alternative aspect, the needle tip is positioned anterior to the vertebral bone without engaging the vertebral bone. The desired result is that the tip of the needle be generally posterior to the carotid sheath exterior surface 106 such that the stimulating electrode lead can be advanced to a position posterior to the carotid sheath.

As shown in FIG. 4A, the needle 710 includes a bevel tip 715 and may include an external flange 723 or other marking device to indicate the orientation of the bevel tip after insertion in the patient. In addition to or as an alternative, the needle tip may be imaged to determine its orientation within the patient. The healthcare provider orients the bevel tip 715 opening generally toward the head and aligned with the carotid sheath exterior surface 106. Once the needle tip has been properly positioned as confirmed by fluoroscopy and the tip oriented in the appropriate direction, an electrode lead assembly 730 is advanced through the interior of the needle. The stabilization member of the lead assembly is compressed to a collapsed configuration to ease insertion through the needle and patient tissue within a delivery sheath 765. As the tip of the lead 718 and delivery sheath 765 exit the needle tip 715 it is directed cephalad along axis L2 as a result of the bevel tip needle opening orientation. Longitudinal axis L2 extends laterally away from longitudinal axis L3 of the insertion needle at an angle less than 90 degrees. The delivery sheath 765 with constrained lead are advanced from the needle until the electrode assembly 730 has passed through the opening of the needle. While maintaining the position of the electrodes 730, the delivery sheath 765 is withdrawn from the patient. Additional fluoroscopic views may be used to confirm electrode placement adjacent the carotid sheath 104 posterior exterior surface 106 proximal to the vagus nerve 100. In one aspect, the electrode assembly 716 is positioned between the longitudinal muscles and the carotid sheath. The longitudinal muscles may be the longus colli or the longus capitis depending on specific patient anatomy and the cervical level where the stimulation electrode is being placed. In one aspect, the electrode assembly is at least partially circumferentially shielded to provide an area of stimulation adjacent electrodes 716′ and an area of insulation adjacent insulating barrier 741. During deployment of the electrode assembly, the surgeon positions the assembly so that the electrodes 716 are oriented toward the posterior side of the vagus nerve 100. To assist with verification of orientation, the left wing 734 includes a radiopaque marker 747. In the A-P fluoroscopic view, if the marker 747 appears to the left of the stimulating electrodes 716′, then the device is properly oriented. Other means of determining the orientation and/or controlling the orientation of the electrodes can be included. For example, but without limitation, the lead body 720′ may include a visual indicator such as a longitudinal stripe along the side with the electrodes 716′. Still further, the lead body 720′ may include a longitudinal projection and the needle may include a guiding keyway to receive the projection and thereby control the orientation of the electrode lead assembly 730 during deployment.

In still a further aspect, the wings 732 and 734 are formed of a resilient material. The wings are compressed in the delivery cannula 765 into a collapsed insertion configuration and resiliently expand to substantially the stabilizing configuration shape shown in FIGS. 7A and 7B upon exiting the delivery cannula 765. In this aspect, the method includes manipulating the expanded electrode assembly 730 to position the electrodes 716 to be proximal the posterior surface 106 of the carotid sheath 104 and as close as possible to the vagus nerve. As an alternative, the method can include maneuvering the electrode assembly 730 to the preferred stimulation position along the posterior of the carotid sheath adjacent the vagus nerve and then deploying the wings to their anchoring and shielding configuration shown in FIG. 4B. The deployment may be by resilient expansion or in the alternative embodiment described above by delivering fluid into a bladder formed within the wings. Once the electrode assembly 730 is positioned, initial stimulation can occur to evaluate the initial position. The assembly can be moved to a better position to stimulate the vagus nerve if necessary to achieve increased stimulation. In the final position, the lead wire 750 is left in position extending out of the patient through opening 713. A bacteriostatic ring can be positioned about lead wire 750 and affixed to the skin to inhibit bacterial infection.

After placement of the temporary electrode assembly 730, various stimulation protocols can be utilized to screen the response of the patient to vagus nerve stimulation as described above. In a further exemplary method, the electrode is removed from the patient after a given trial period of screening. In one aspect, the electrode assembly is removed from the patient by pulling on the lead wire 750 to dislodge the electrodes and securing assembly. In an alternative approach, a tubular retrieval instrument, similar to delivery sheath 765, is advanced along the lead wire 750. As the end of the retrieval instrument engages the trailing tapers 736 and 738, the wings will be collapsed inside the retrieval instrument. Continued advancement of the tubular retrieval instrument toward the distal end 718′ of the lead will completely collapse the winged assembly and position it inside the retrieval instrument. After the wings are collapsed and inside the retrieval instrument, the instrument with included lead can be withdrawn from the patient.

If screening of vagus nerve stimulation during the trial period provided a beneficial effect, the method may further include placement of a permanent electrode assembly and an implantable pulse generator similar to the system of FIG. 1. In a similar fashion, stimulation capsules with self contained pulse generators may be positioned to permanently stimulate the vagus nerve.

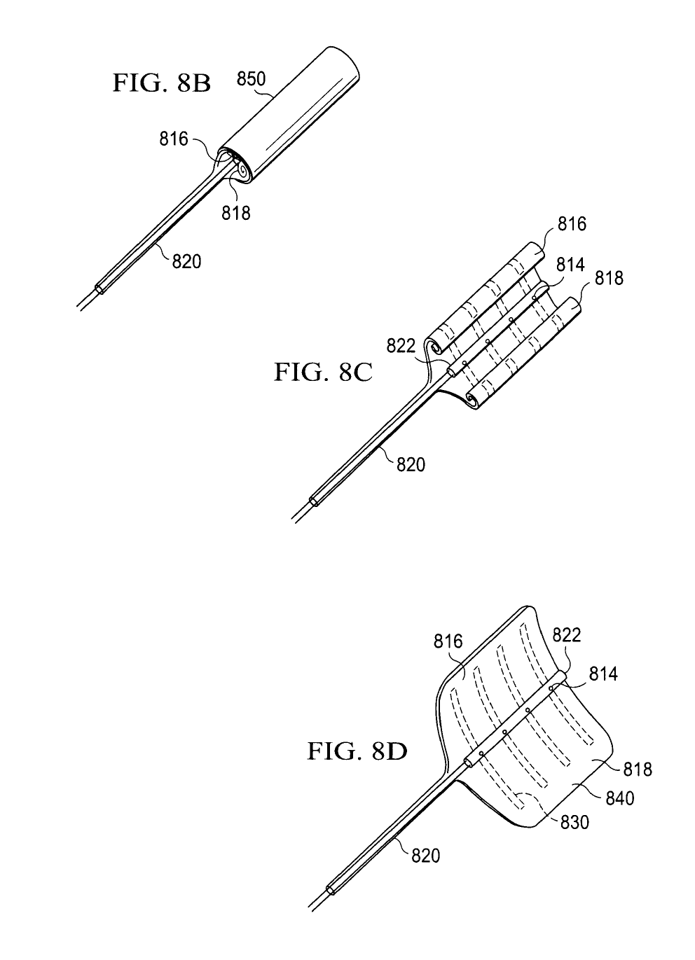

Referring now to FIG. 8A, there is shown a further embodiment of a temporary electrode assembly 800. The assembly 800 includes a lead body 820 and an electrode array head assembly 810. The head assembly 810 includes a series of electrodes 814 spaced along the lead body. A series of stiffening members 830 are disposed adjacent to the electrodes and extend substantially transverse to the longitudinal axis of the lead body. The stiffening members are resilient and tend to return to the shape shown in FIGS. 8A and 8D. A coating 840 of insulating material encapsulates the stiffening members and attaches to the back side of the lead body, opposite the electrodes 814. The coating 840 in combination with the stiffening members 830 forms wings 816 and 818 that extend laterally from the lead body 820. As shown in FIG. 8B, the wings 816 and 818 may be rolled so they form a reduced sized insertion configuration adapted to be received in cylindrical delivery cannula 850. The delivery cannula 850 maintains the head assembly in the insertion configuration shown in FIG. 8B until the distal end 822 exits the distal end of the delivery cannula. As shown in FIG. 8C, the stiffening members tend to expand the wings 816 and 818 once they have passed out of the delivery cannula. Once fully unconstrained, the stiffening members 830 urge the wings 816 and 818 into the anchoring and shielding configuration shown in FIGS. 8A and 8D. As best illustrated in FIG. 8D, the stiffening members define an inner concave surface adjacent the electrodes 814 and an outer concave surface opposite the electrodes. It is contemplated that such a curved configuration more closely matches the posterior carotid sheath anatomy in the preferred stimulation area. Use of assembly 800, in at least one method of placement, is consistent with that described above for lead assembly 712′.

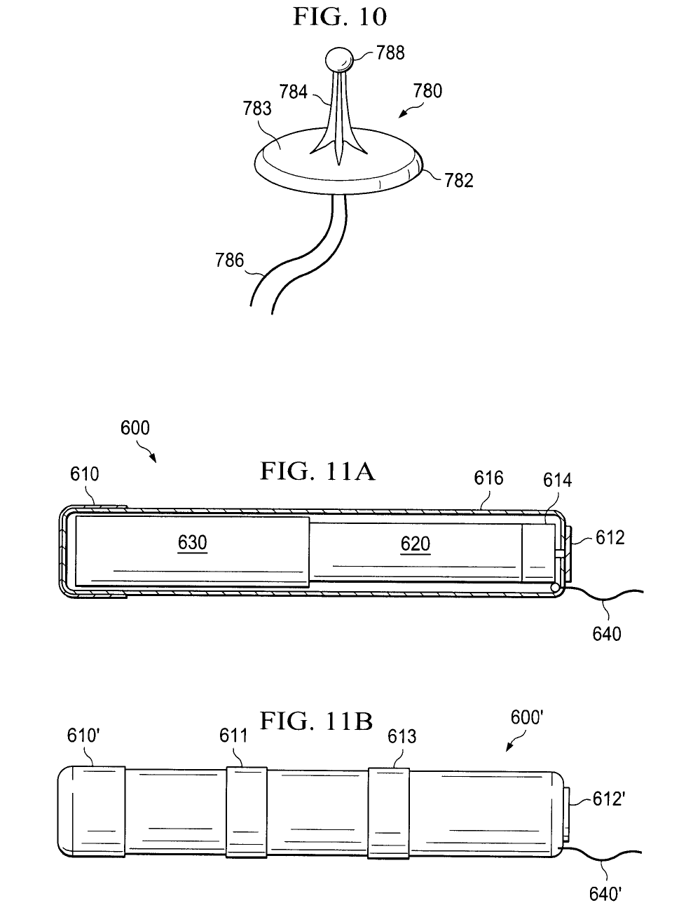

Referring now to FIG. 10, there is shown still a further temporary electrode assembly 780. Electrode assembly 780 includes a body 782 defining a skin engaging surface 783 which may, in at least one embodiment, include an adhesive coating to adhere to the skin. A stalk 784 is supported on body 782 extending away from surface 783. A stimulation electrode 788 is disposed on the distal end of the stalk 784 and is electrically connected to lead wire 786 that extends internally to the stalk. In use, the stimulation electrode is brought into initial contact with the skin of the patient with a first distance between the temporary electrode and the vagus nerve. The method continues with forcing the electrode and stalk through the patient's skin in a direction toward the vagus nerve to a second position with a second distance between the temporary electrode and the vagus nerve, the second distance being less than the first distance. An instrument may be used to assist with piercing the skin if necessary. It is contemplated that once the electrode is in the desired stimulation area the surface 783 engages the adjacent skin. If desired tape alone or in combination with a compression pad over the lead body may be used to apply compression to the lead to urge it to maintain the position closer to the vagus nerve. It is contemplated that in one exemplary form of the method the electrode is placed within 1 cm of the vagus nerve. With the electrode properly positioned, lead wire 786 is then connected to a pulse generator and stimulation is conducted to trial electrical stimulation response on the nerve adjacent the electrode.

In a further aspect of the present disclosure, capsules or microstimulators 600 or 600′ such as shown in FIGS. 11A and 11B can be placed percutaneously near the vagus nerve or in the pocket formed between the skin 101 and the carotid sheath 104 described above. The bipolar microstimulator 600 of the present disclosure is similar to or of the type referred to as BION devices. The microstimulator 600′ is similar to the bipolar version shown in FIG. 11A but includes four electrodes 610′, 611, 613 and 612′. Although not required for use, it is contemplated that each of the capsule stimulators 600 and 600′ are joined to tethers 640 and 640′ respectively. For temporary electrode placement associated with the disclosed trialing techniques, it may be desirable to be able to pull on the tethers 640/640′ to remove the stimulators without gaining surgical access to grasp the capsules with an instrument. It is contemplated that the tethers 640/640′ can be left just under the skin after capsule implantation for easy access for capsule removal at a later date. This can be particularly advantageous when placing the capsules adjacent the posterior carotid sheath surface, as this area is more difficult to access for surgical removal of the capsules.

The following documents describe various features and details associated with the manufacture, operation, and use of BION implantable microstimulators, and are all incorporated herein by reference: U.S. Pat. Nos. 5,193,539; 5,193,540; 5,312,439; 5,324,316; 5,405,367; 6,051,017 and PCT Publications WO 98/37926; WO 98/43700; WO 98/43701.

As shown in FIG. 11A, microstimulator device 600 includes a narrow, elongated capsule 616 containing electronic circuitry 620 connected to electrodes 610 and 612, which may pass through the walls of the capsule at either end. As detailed in the referenced patent publications, electrodes 610 and 612 generally comprise a stimulating electrode (to be placed close to the nerve) and an indifferent electrode (for completing the circuit). Other configurations of microstimulator device 600 are possible, as is evident from the above-referenced publications, and as described in more detail herein.

Certain configurations of implantable microstimulator 600 are sufficiently small to permit its placement adjacent to the structures to be stimulated. (As used herein, “adjacent” and “near” mean as close as reasonably possible to the target nerve, including touching or even being positioned within the target nerve, but in general, may be as far as about 150 mm from the target nerve.) A single microstimulator 600 may be implanted, or two or more microstimulators may be implanted to achieve greater stimulation of the nerve fibers, or for a longer period of time.

Capsule 616 of FIG. 11A may have a diameter of about 4-5 mm, or only about 3 mm, or even less than about 3 mm. Capsule 152 length may be about 25-35 mm, or only about 20-25 mm, or even less than about 20 mm. The shape of the microstimulator may be determined by the structure of the desired target, the surrounding area, and the method of implantation. A thin, elongated cylinder with electrodes at the ends, as shown in FIG. 11A, is one possible configuration, but other shapes, such as spheres, disks, or helical structures, are possible, as are additional electrodes similar to the configuration shown in FIG. 11B.

Microstimulator 600 may be implanted with a surgical insertion tool specially designed for the purpose, or may be placed, for instance, via a small incision and through an insertion cannula as has been previously described above. Alternatively, device 600 may be implanted via conventional surgical methods, or may be inserted using other endoscopic or laparoscopic techniques. A more complicated surgical procedure may be required for sufficient access to a nerve or a portion of a nerve (e.g., nerve fibers surrounded by scar tissue, or more distal portions of the nerve) and/or for fixing the neurostimulator in place. As explained above, tether 640 may be included with capsule 616 to allow the temporary trialing electrode to be removed without gaining renewed surgical access to the stimulation site.

The external surfaces of stimulator 600 may advantageously be composed of biocompatible materials. Capsule 616 may be made of, for instance, glass, ceramic, or other material that provides a hermetic package that will exclude water vapor but permit passage of electromagnetic fields used to transmit data and/or power. Electrodes 610 and 612 may be made of a noble or refractory metal or compound, such as platinum, iridium, tantalum, titanium, titanium nitride, niobium, or alloys of any of these, in order to avoid corrosion or electrolysis which could damage the surrounding tissues and the device.

In certain embodiments of the present disclosure, microstimulator 600 comprises two, leadless electrodes. However, either or both electrodes 610 and 612 may alternatively be located at the ends of short, flexible leads as described in U.S. patent application Ser. No. 09/624,130, filed Jul. 24, 2000, which is incorporated herein by reference in its entirety. The use of such leads permits, among other things, electrical stimulation to be directed more locally to a specific nerve structure(s) a short distance from the surgical fixation of the bulk of the implantable stimulator 600, while allowing most elements of stimulator 600 to be located in a more surgically convenient site. This minimizes the distance traversed and the surgical planes crossed by the device and any lead(s). In most uses of this disclosure, the leads are no longer than about 150 mm.

Microstimulator contains, when necessary and/or desired, electronic circuitry 620 for receiving data and/or power from outside the body by inductive, radio-frequency (RF), or other electromagnetic coupling. In some embodiments, electronic circuitry 620 includes an inductive coil 614 for receiving and transmitting RF data and/or power, an integrated circuit (IC) chip for decoding and storing stimulation parameters and generating stimulation pulses (either intermittent or continuous), and additional discrete electronic components required to complete the electronic circuit functions, e.g. capacitor(s), resistor(s), coil(s), and the like.

Neurostimulator 600 includes, when necessary and/or desired, a programmable memory for storing a set(s) of data, stimulation, and control parameters. Among other things, memory may allow stimulation and control parameters to be adjusted to settings that are safe and efficacious with minimal discomfort for each individual. Specific parameters may provide therapeutic advantages for various medical conditions, their forms, and/or severity. For instance, some patients may respond favorably to intermittent stimulation, while others may require continuous stimulation to alleviate their symptoms.

In addition, stimulation parameters may be chosen to target specific neural populations and to exclude others, or to increase neural activity in specific neural populations and to decrease neural activity in others. For example, relatively low frequency neurostimulation (i.e., less than about 50 100 Hz) typically has an excitatory effect on surrounding neural tissue, leading to increased neural activity, whereas relatively high frequency neurostimulation (i.e., greater than about 50 100 Hz) may have an inhibitory effect, leading to decreased neural activity.

Some embodiments of implantable stimulator 600 also include a power source and/or power storage device 600. Possible power options for a stimulation device of the present disclosure, described in more detail below, include but are not limited to an external power source coupled to stimulator 600, e.g., via an RF link, a self-contained power source utilizing any suitable means of generation or storage of energy (e.g., a primary battery, a replenishable or rechargeable battery such as a lithium ion battery, an electrolytic capacitor, a super- or ultra-capacitor, or the like), and if the self-contained power source is replenishable or rechargeable, means of replenishing or recharging the power source (e.g., an RF link, an optical link, a thermal link, or other energy-coupling link).

According to certain embodiments of the present disclosure, a microstimulator operates independently. According to various embodiments of the present disclosure, a microstimulator operates in a coordinated manner with other microstimulator(s), other implanted device(s), or other device(s) external to the patient's body. For instance, a microstimulator may control or operate under the control of another implanted microstimulator(s), other implanted device(s), or other device(s) external to the patient's body. A microstimulator may communicate with other implanted microstimulators, other implanted devices, and/or devices external to a patient's body via, e.g., an RF link, an ultrasonic link, a thermal link, an optical link, or the like. Specifically, a microstimulator may communicate with an external remote control (e.g., patient and/or physician programmer) that is capable of sending commands and/or data to a microstimulator and that may also be capable of receiving commands and/or data from a microstimulator.

Applicants note that the procedures disclosed herein are merely exemplary and that the systems and methods disclosed herein may be utilized for numerous other medical processes and procedures. Although several selected embodiments have been illustrated and described in detail, it will be understood that they are exemplary, and that a variety of substitutions and alterations are possible without departing from the spirit and scope of the present invention, as defined by the following claims.

We claim:

1. A method of screening vagus nerve stimulation to determine efficacy, the method comprising:
placing at least one temporary electrode adjacent the vagus nerve;
energizing the electrode to stimulate at least a portion of the vagus nerve;
monitoring patient response to stimulation of the vagus nerve;
determining whether vagus nerve stimulation had a beneficial effect on the patient condition; and
removing the temporary electrode.

2. The method of claim 1, wherein said placing includes positioning the temporary electrode on the skin of a patient.

3. The method of claim 2, wherein said securing includes adhering at least a portion of the electrode to the skin of a patient.

4. The method of claim 1, wherein said placing includes making initial contact between the patient and the temporary electrode and advancing the temporary electrode toward the vagus nerve.

5. The method of claim 4, wherein the initial contact is on the skin of the patient with a first distance between the temporary electrode and the vagus nerve; and the advancing includes pushing the temporary electrode through the skin in a direction toward the vagus nerve to a second position with a second distance between the temporary electrode and the vagus nerve, the second distance being less than the first distance.

6. The method of claim 5, wherein said securing includes applying a compression member to maintain the electrode in the second position.

7. The method of claim 1, wherein said placing includes forming an opening in the skin of the patient and positioning the temporary electrode beneath the skin of the patient.

8. The method of claim 7, wherein said positioning includes:
performing a blunt needle stick adjacent the carotid sheath of the patient;
imaging a portion of the electrode to monitor the electrode position within the patient;
advancing the temporary electrode along the carotid sheath substantially parallel to the vagus nerve; and
securing the temporary electrode.

9. The method of claim 8, wherein said advancing includes moving the temporary electrode along the exterior of the carotid sheath.

10. The method of claim 8, wherein said advancing includes moving the temporary electrode within the interior of the carotid sheath.

11. The method of claim 8, wherein said advancing includes providing a blunt catheter, advancing the blunt catheter along the carotid sheath, the electrode associated with the blunt catheter to position the electrode substantially parallel to the vagus nerve.

12. The method of claim 1, wherein said placing includes positioning the electrode adjacent the posterior of a carotid sheath.

13. The method of claim 7, wherein said positioning includes:
creating an incision above the clavicle;
forming a pocket parallel to and outside of the carotid sheath of the patient;
inserting a temporary lead having at least two electrodes disposed thereon; and
positioning the temporary lead electrodes to engage at least a portion of the carotid sheath facing the vagus nerve.

14. The method of claim 13, wherein the temporary lead has an insertion configuration and a stimulating configuration, the method further including inserting the temporary lead into the patient in the insertion configuration and positioning the temporary lead electrodes to extend about at least a portion of the carotid sheath by deploying the temporary lead from the insertion configuration to the stimulating configuration.

15. The method of claim 14, wherein the securing step is performed at least in part by deploying the temporary lead from the insertion configuration to the stimulating configuration.

16. The method of claim 7, wherein the electrode includes a lead and said anchoring includes adhering at least a portion of the lead to the skin of the patient adjacent the opening.

17. The method of claim 7, wherein the electrode is connected to an implantable stimulator and said securing includes closing the opening to inhibit the stimulator from exiting the patient.

18. The method of claim 1, further including stimulating the temporary electrode for a predetermined period; monitoring patient seizure activity during the stimulation period; comparing the patient response to a baseline of patient seizure activity without stimulation to the patient seizure activity during the stimulation period.

19. The method of claim 1, further including energizing the temporary electrode prior to said securing, monitoring patient response to observe effective stimulation and securing the temporary electrode in a position to cause vagus nerve stimulation when the electrode is energized.

20. The method of claim 18, wherein the stimulating is substantially continuous during the predetermined period.

21. The method of claim 18, wherein the stimulating occurs according to a predetermined cycle during the predetermined period.

22. The method of claim 18, further including controlling the energizing to occur at least during seizures.

23. The method of any of the proceeding claims 1-22, further including after said removing the temporary electrode, placing a permanent electrode to stimulate the vagus nerve and energizing the electrode with an implantable pulse generator.

24. A system for screening of the vagus nerve for stimulation efficacy, comprising:
a base member with a longitudinal axis and having a first side with at least one electrode and an opposing second side with a non-conductive surface;
an electrically conductive lead extending within said base and electrically connected to said electrode; and
a stabilization member extending laterally away from the longitudinal axis adjacent the at least one electrode, the stabilization member for maintaining the electrode adjacent the vagus nerve and the non-conductive surface away from the vagus nerve.

25. The system of claim 24, wherein the stabilization member includes at least one wing extending laterally between the first side and the opposing second side.

26. The system of claim 24, wherein the stabilization member has a collapsed configuration for insertion to a nerve stimulation site and an expanded stabilizing configuration.

27. The system of claim 26, wherein the stabilization member resiliently expands from the collapsed configuration to the expanded stabilizing configuration.

28. The system of claim 27, wherein the stabilization member further includes at least one stiffening member.

29. The system of claim 24, wherein the first side is substantially concave.

30. The system of claim 26, wherein the stabilization member includes at least one internal fluid bladder, whereby fluid injected into the bladder moves the stabilization member between the collapsed configuration and the expanded stabilization configuration.

31. A kit for screening the vagus nerve for stimulation efficacy through a minimally invasive surgical approach, the kit comprising:
at least one temporary vagus nerve electrode assembly configured for unattached stimulation of the vagus nerve, and
an access needle configured for gaining surgical access through a patient's skin and having an internal bore sized to pass the at least one temporary vagus nerve electrode assembly.

32. The kit of claim 31, further including a guide wire and dilating sheath.

33. The kit of claim 31, further including a bacteriostatic ring and a screening cable.
